(19) United States
(12) Patent Application Publication
(10) Pub. No.: US 2026/0286272 A1
(43) Pub. Date: Sep. 24, 2026

(54) ADJUSTABLE LOADING DEVICE

(71) Applicant: DESICORP INC., Louisville, KY (US)

(72) Inventors: Brett JANIS, Louisville, KY (US); James WELCH, Louisville, KY (US); Emily MURPHY, Louisville, KY (US)

(21) Appl. No.: 19/478,299

(22) PCT Filed: Apr. 25, 2024
(86) PCT No.: PCT/US2024/026246

§ 371 Date: Oct. 24, 2025

Related U.S. Application Data
(60) Provisional application No. 63/461,746, filed on Apr. 25, 2023.

(51) Int. Cl. C12M 1/42; A61B 5/15; C12M 1/26

(52) U.S. Cl. CPC C12M 35/04; C12M 33/18; C12M 35/02; C12M 35/06; A61B 5/150755

(57) ABSTRACT

An adjustable loading device is provided. In one embodiment, the adjustable loading device is an acoustofluidic loading device that includes a bottom platform having a plurality of rollers and a movable section; wherein the movable section is configured to move from at least a first position to a second position and wherein at least one of the plurality of rollers is fixedly connected to the movable section. The loading device may also include a portion of tubing having an elongated lumen therethrough; wherein the portion of tubing is configured to extend around or about the plurality of rollers on the bottom platform to create an adjustable tubing path; and an upper platform disposed above the bottom platform and including an energy emitting device that is positioned to emit energy toward at least a portion of the tubing path.

### TECHNICAL FIELD

The present disclosure relates to an adjustable loading device for use in delivering molecular payloads, such as cryoprotective agents, proteins, genetic materials, or other molecules, into the interior compartments of cells. More specifically, the present disclosure relates to an adjustable acoustofluidic, electrofluidic, or magnetofluidic enabled cell loading device.

### BACKGROUND

One of the ongoing challenges of cryobiology is the transportation of cryoprotective agents (CPAs) into the interior compartments of cells. Some CPAs are membrane-permeant, such as glucose, but these compounds tend to be metabolizable by the cells or are limited to smaller molecules. Larger CPAs, or CPAs that are derived from different biological or chemical backgrounds, are often membrane-impermeant. This makes it difficult to protect proteins and DNA within the cytosol or within various organelles. It would be beneficial to provide a method of effectively loading CPAs into the interior compartments of cells without damaging the cell membrane. Furthermore, many cryoprotective proteins are well established in nature, but genetically modifying cells to express them is challenging and limits their therapeutic use. Loading proteins directly reduces the restrictions of cells for therapeutic use, but it also requires very precise optimization of loading parameters.

### SUMMARY

An adjustable loading device is provided. In one embodiment, the adjustable loading device is an acoustofluidic loading device. In one embodiment the adjustable loading device includes a bottom platform having a plurality of rollers and a movable section; wherein the movable section is configured to move from at least a first position to a second position and wherein at least one of the plurality of rollers is fixedly connected to the movable section. The loading device may also include a portion of tubing having an elongated lumen therethrough; wherein the portion of tubing is configured to extend around or about the plurality of rollers on the bottom platform to create an adjustable tubing path; and an upper platform disposed above the bottom platform and including an energy emitting device that is positioned to emit energy toward at least a portion of the tubing path. In one embodiment, the energy emitting device is an ultrasound device capable of emitting ultrasound energy/waves (in one case having a frequency greater than about 20 kHz, and in another case greater than about 100 kHz, and in another case greater than about 1 MHz and in one case less than about 1 GHz, and in another case less than about 5 GHz, and in yet another case less than about 10 MHz).

In another embodiment, the tubing path has a shape that is defined by the portion of tubing extending between the plurality of rollers and is configured to have a first length and a first shape when the movable section of the bottom platform is in the first position and a second length and a second shape when the movable section of the bottom platform is in the second position. The tubing path may be configured to have a first shape that is a spiral, zig-zag, elongated oval shape, S-shape, or a combination thereof. In one embodiment, first length is less than the second length.

The device may further include at least one threaded pillar configured to connect the bottom platform to the upper platform, wherein the upper platform is configured to be movable toward and away from the bottom platform by adjusting the at least one threaded pillar, which may be connected to a motor that rotates the threaded pillar in a clockwise and counterclockwise direction.

In one embodiment, the loading device further includes a first spooling member disposed a first end of the tubing path and a second spooling member disposed at a second end of the tubing path; wherein the first and second spooling members are configured to maintain tension of the portion of tubing that extends along the tubing path. The device may also include a heating or cooling element configured to heat the portion of tubing extending along the tubing path.

In one embodiment, the movable section of the bottom platform is disposed toward a center portion of the bottom platform and is configured to rotate in a clockwise and counter-clockwise direction. In another embodiment, the plurality of rollers includes the at least one roller fixedly connected to the movable section and a second roller fixedly connected to the movable section. And, in yet another embodiment, the movement of the movable section from the first position to the second position is configured to create a spiral shape of the tubing path.

### BRIEF DESCRIPTION OF THE DRAWINGS

FIG. 1 is a schematic view of one example of a method of loading therapeutic compounds into red blood cells using sonoporation:

FIG. 2 is an exploded bottom perspective view of one embodiment of an adjustable loading device;

FIG. 3 is an exploded side perspective view of the top and bottom platforms of the adjustable loading device of FIG. 2;

FIG. 4 is an exploded top view of the adjustable loading device of FIG. 2;

FIG. 5 is a top section view of the bottom platform of the adjustable loading device in a first position, showing a first tubing path;

FIG. 6 is a top section view of the bottom platform of the adjustable loading device in a second position, showing a second tubing path;

FIG. 7 is a top section view of another embodiment of the adjustable loading device;

FIGS. 8 and 9 are schematics showing example of an automated program (FIG. 8) configured to be used with a system including the adjustable loading device (FIG. 9);

FIG. 10 is a top section view of another embodiment of the bottom platform of the adjustable loading device in a first position, showing another embodiment of the tubing path;

FIG. 11 is a top section view of another embodiment of the bottom platform of the adjustable loading device in a second position, showing another embodiment of the tubing path;

FIG. 12 is a top section view of another embodiment of the bottom platform of the adjustable loading device in a first position, showing another embodiment of the tubing path; and

FIG. 13 is a top section view of another embodiment of the bottom platform of the adjustable loading device in a second position, showing another embodiment of the tubing path.

### DETAILED DESCRIPTION

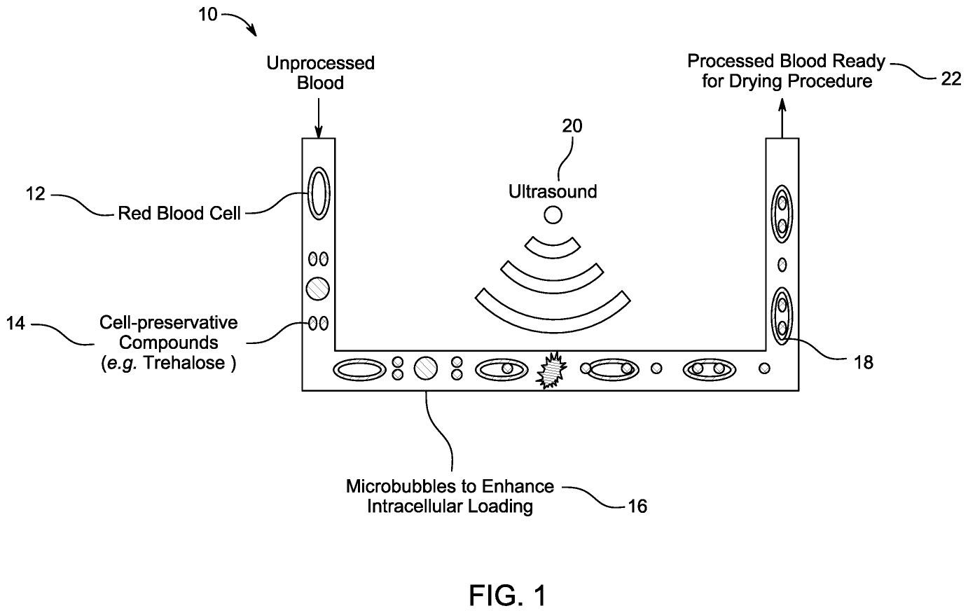

In one embodiment, a high flow acoustofluidic loading platform (HFAL) is used to load CPAs into cells. One such HFAL is disclosed in U.S. patent application Ser. No. 16/622,361 (the entire disclosure of which is incorporated herein). With reference to FIG. 1, this method 10 flows confluent cells 12, such as red blood cells, mixed with osmolytes 14, including CPAs, and also mixed microbubbles 16, through a fluidic chip 18 (i.e. chip, microfluidic chip, fluidic chip, which can meter desired amounts of, for example, microbubbles and/or target agent (preservation agent in one case) into the cell solution) and the cell solution is then exposed to ultrasound waves 20 within the resonance frequency of the microbubbles, as shown in FIG. 1. In one embodiment, the ultrasound device(s) is positioned above, below, or about the flow of the mixture of cells 12, osmolytes 14, and microbubbles 16, such that the energy from the device transverses the flow of the mixture. The ultrasound waves 20 cause the microbubbles 16 to oscillate, eventually resulting in cavitation that produces directional microjets of the surrounding fluid medium that penetrate the membrane of the cells 12.

The microjets cause holes or cavities to be temporarily formed in the cell membranes. Dissolved osmolytes 14, including CPAs such as trehalose or proline, are thereby forcibly injected and/or naturally flow into the cell 12 through the holes or cavities. The cell membrane is then rapidly repaired by cell processes, closing the holes or cavities and leaving the osmolytes, or a portion thereof, disposed within the cell 12. It has been found that the cell 12 can evacuate membrane-permeant compounds through passive or active transport mechanisms, but membrane-impermeant compounds can remain inside of the cell 12 where they can offer protection to intracellular targets, producing treated cells 22. This process occurs as the mixture is flown through a fluidic chip 18, which can be as smaller than two inches. Multiple fluidic chips can be utilized in parallel, or in sequence, to increase loading throughput to extremely high rates with minimal space and power footprints.

This active loading means that cells 12 can be repeatedly loaded with different compounds, which is difficult to accomplish with diffusion-based methods, such as electroporation. The use of a propagating energy source like an ultrasound device to generate microjets facilitates a large flow channel size (i.e. 0.1 to about 5 millimeters in diameter) used for this method and allows a larger volume of cells to be loaded with compounds even in mixtures having high cell concentrations.

A variety of parameters including flow rate, ultrasound frequency, ultrasound pressure, ultrasound duration, microbubble size, microbubble charge, microbubble dose, microbubble composition, sheer force, inertial focusing, temperature, tubing diameter, and buffer composition can all be optimized for specific loading applications. This allows the user to rapidly load large payloads exceeding 40 kDa into both sensitive and robust cells. This loading method may be used to load osmolytes, polymers, proteins, and nucleic acids into red blood cells and other nucleated cells from several taxonomic groups including mammals, insects, and bacteria.

However, different cells may require different loading parameters, which may necessitate the generation of new fluidic chip designs. Additionally, in existing designs, certain surfaces of the fluidic chip must be compatible with the cells and buffer components because the mixture, including the cells, contact portions of the chip during loading. Thus, in many existing designs, in the case of treating human red blood cells, each microfluidic chip will need to be washed and sterilized between every blood unit. These same steps may be required for switching between any other medical cell suspension or for sterile research cell lines. Some existing chips may be disposable if the material was selected for both its compatibility and its affordability, but it is beneficial to use adjustable durable chips that are able to be cleaned and reused, especially if multiple chips are to be used for each unit of cells.

In order to address the challenges described above, an adjustable high flow loading device is provided, which may be used, in some embodiments, to replace existing fluidic chip designs. In one embodiment, the adjustable loading device is an adjustable high flow acoustofluidic loading device (hereinafter “adjustable HFAL”). It should be understood that the high flow loading device may also be an electrofluidic, magnetofluidic, or other suitable loading device. In some embodiments, and as described in more detail below, the adjustable loading device may include 1) dry-coupled or ultrasound gel-coupled signal transduction between an ultrasound source and a fluidic mixture flowing through a portion of round or square tubing, 2) an adjustable surface that creates a desired flow path by manipulating tubing into non-linear shapes such as, but not limited to, concentric spirals or zig zags, a clamping mechanism or a vise mechanism to ensure full ultrasound treatment of the cells, 3) a Peltier heating or cooling system to control the temperature of the mixture inside the tubing, 4) tension rollers to maintain a consistent tubing shape, and 5) an in-line microbubble mixing site or the ability to load samples where the microbubbles are premixed within the sample.

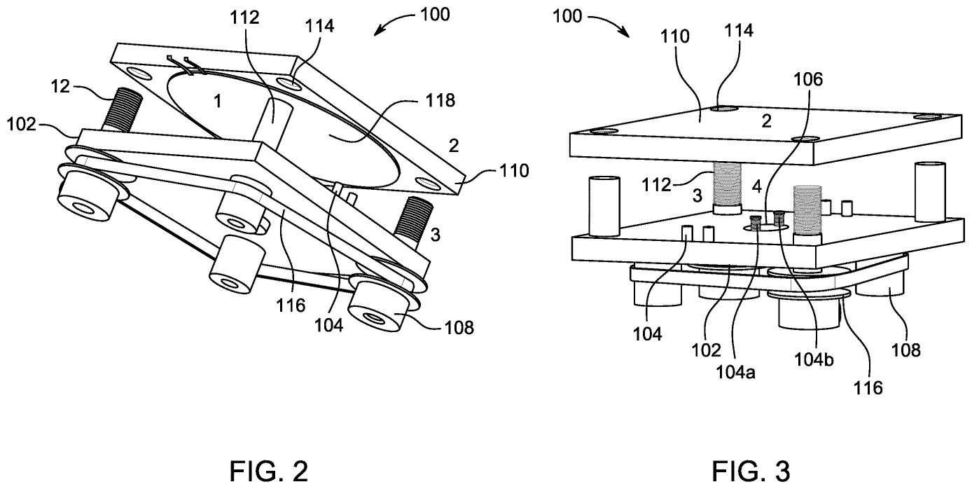

Referring now to FIGS. 2 and 3, in one embodiment, the adjustable loading device 100 includes a bottom platform 102 with a generally flat, planar upper surface. The loading device 100/bottom platform 102 can also include at least one movable section 106 positioned in the platform 102, where the movable section 106 has a generally flat, planar upper surface that is flush/aligned with the upper surface of the bottom platform 102. The movable section 106 may be generally circular in top view, and is configured to be closely received in a correspondingly-shaped recess formed in or through the bottom platform 102. The movable section 106 can have a plurality of protrusions/rollers 104a, 104b protruding upwardly/outwardly from or through the upper surface of the movable section 106. The bottom platform 102 may further include a set of feet 108 positioned on an opposite side relative to the upper surface, configured to support the adjustable loading device 100. In one embodiment, the movable section 106 is disposed at or toward the center of the bottom platform 102 and is configured to rotate/move from at least a first position to a second position, such that the protrusions/rollers 104a, 104b are termed movable protrusions/rollers.

In one embodiment, the movable section 106 can rotate in a partial or full revolution (360 degrees) or multiple revolutions or fractions thereof. In another embodiment (not shown), the protrusions/rollers 104a, 104b may be designed to move about the bottom platform 102 in a linear and/or non-rotational manner. For example, the movable protrusions 104a, 104b may be moved in opposite linear directions, or the same linear directions (but different extents, in one case), or perpendicular to each other, or one may be moved linearly while the other is moved along an arcuate path, or one is moved while the other remains stationary, etc.

Moreover, in one embodiment, at least one, and preferably two, of the plurality of rollers (movable rollers 104a and 104b) are fixedly connected to the movable section 106 and positioned on opposite sides thereof. Furthermore, at least one, and preferably two of the rollers (fixed rollers 104c and 104d) are fixedly connected to the bottom platform 102 and positioned on opposite sides thereof, and not positioned on the movable section 106. Each roller/protrusion 104 can have a rotatable sleeve or the like on an outer surface thereof which is configured to roll when tubing 120 is moved thereacross; however, each roller/protrusion may lack the rotatable sleeve and take the form of a simple protrusion, in which case the roller/protrusion may have a low friction and/or pliable outer surface to accommodate sliding and avoid damage to the tubing 120.

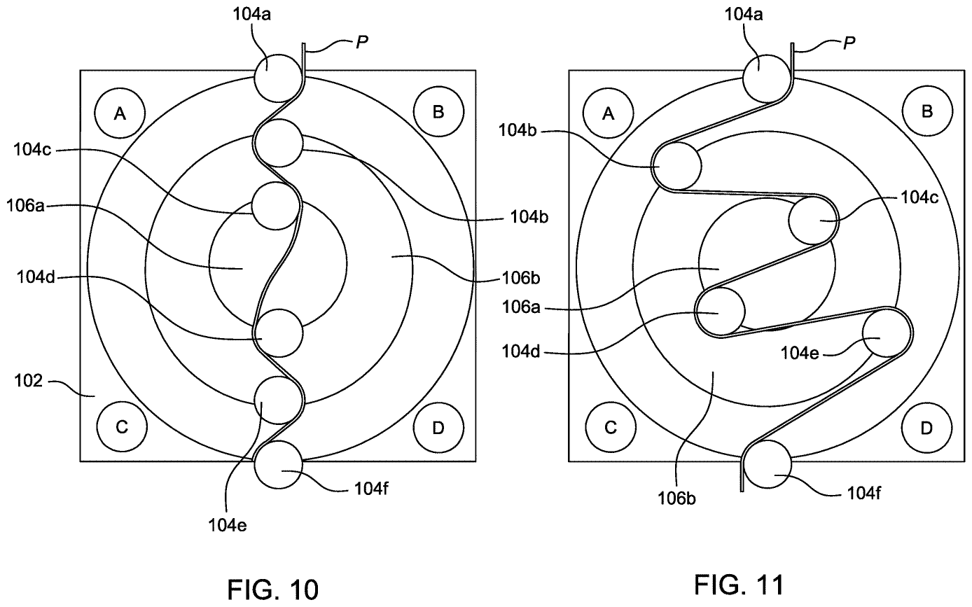

In another embodiment, as shown in FIGS. 10 and 11, the bottom platform 102 may include more than one movable sections (106a and 106b). In this embodiment, each movable section 106a, b, c, etc., may be configured to include one or more rollers or protrusions 104 and move independently from one another relative to the bottom platform 102, allowing the user to create custom flow paths (P), depending upon the desired effect on the cells in use in the device.

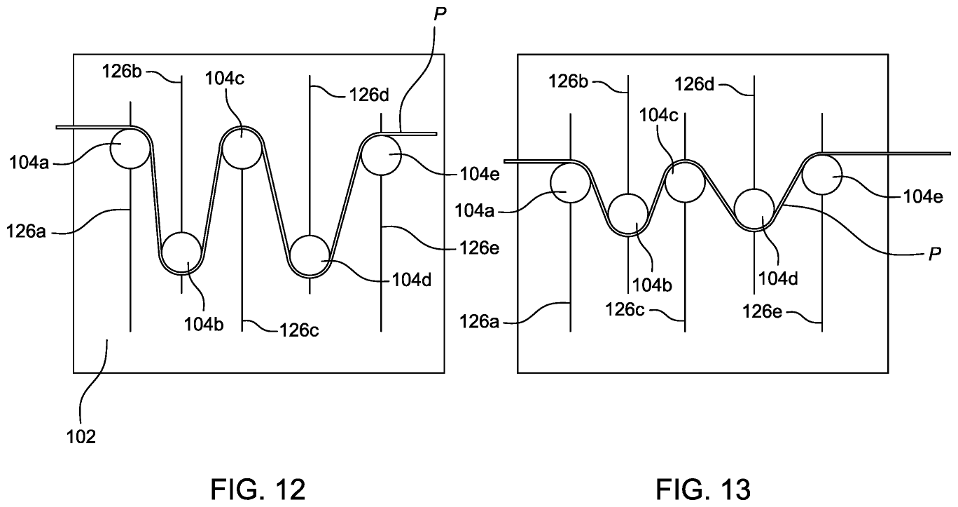

In yet another embodiment, as shown in FIGS. 12 and 13, the bottom platform 102 may include slots 126, rather than movable sections, through which the protrusions 104 may be configured to move. The protrusions 104a, b, c, d, e, etc., may extend through the slots and may be manually or mechanically adjustable to form customizable flow paths (P).

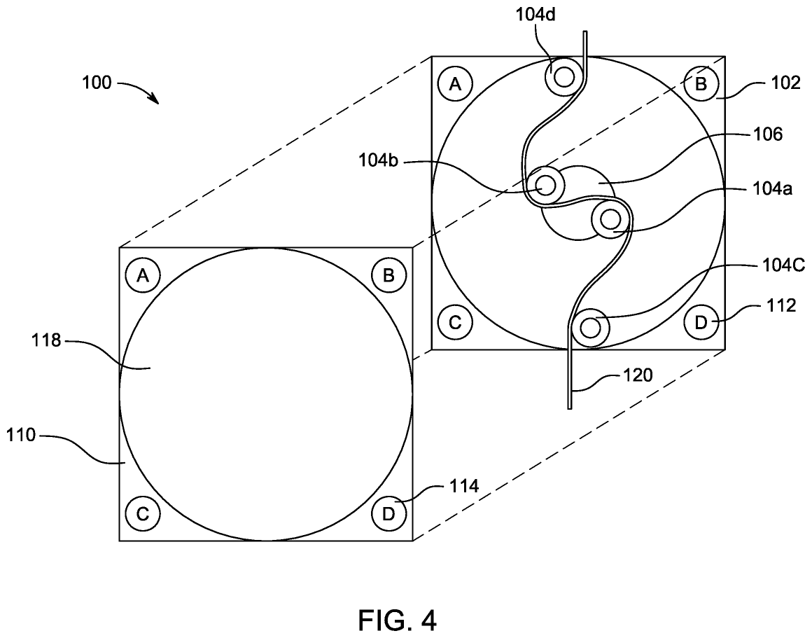

In one embodiment, the adjustable loading device 100 may also include an upper platform 110 disposed above, and oriented generally parallel to the bottom platform 102 by a set of threaded pillars 112 extending therebetween. The upper platform 110 can have a generally flat, planar lower surface thereof, and the upper platform 110 and bottom platform 102 thereby define a working space therebetween. In one embodiment, the threaded pillars 112 are threaded through holes in each corner of the bottom platform 102 and are configured to connect the bottom platform 102 to the upper platform 110, by for example, threading the pillars 112 within holes 114 disposed at corresponding corners of the upper platform 110 (see also FIG. 4).

In one embodiment, the pillars 112 enable the movement of the upper platform 110 toward and away from the bottom platform 102 by adjusting the at least one threaded pillar 112, which may be connected to a motor 116 that rotates the threaded pillars 112 in a clockwise and/or counterclockwise direction. In another embodiment, the pillars 112 may be adjusted manually. This motor can be attached to one of the threaded pillars directly, or interface with one or more pillars through a belt, gear system, or some other method of transferring motion to change the distance between the upper and lower platforms. By adjusting the pillars 112, the upper platform 110 may be used to adjust a height of the working space, to thereby apply or relieve pressure on, or prevent rolling of the, the tubing 120 by the lower surface of the upper platform. However, a distance between the upper platform 110 and bottom platform 102 (and thereby a height of the working space) can be varied or adjusted by any of a wide variety of other means or mechanisms, such as by hydraulic lifts, scissors-type lifts, compressible springs, etc. Moreover, it should be understood that the loading device 100 may not necessarily include the upper platform 110, such as when it is not needed to clamp the tubing 120 or house the energy emitting device 118

In one embodiment, the upper platform 110 may further include or be coupled to an energy emitting device 118 that is configured and positioned to emit energy toward the working space, and thereby toward at least a portion of the tubing path positioned in the working space. In one embodiment, the energy emitting device 118 may be an ultrasound device capable of emitting ultrasound waves. If using an ultrasound device, it may be beneficial to include additional agents, such as coupling gels or structures to enhance the ultrasound emission. However it should be understood that any device capable of emitting an amount of energy, such as an electric current or a magnetic field, may be suitable for use in the loading device 100. Typically, the energy will be suitable for generating a resonance frequency of the microbubbles.

The energy emitting device 118 may be generally disposed on or within the bottom surface of the upper platform 110 and positioned such that the energy emitted from the energy emitting device 118 is directed in a downward direction toward at least a portion of an upper surface of the lower platform 102 and/or toward the working space. In some cases, if desired one or all of the protrusions 104 can be coupled to the upper platform 110 and extend downwardly therefrom, such that a distal end of the protrusions 104 is positioned adjacent to the upper surface of the bottom platform 102 and positioned to engage and interact with tubing 120 positioned on the bottom platform 102. In another embodiment, the upper platform 110 does not include an energy emitting device 118, but is used to simply squeeze the portion of tubing 120 (described below) within the flow path (P) to create the desired effect on the target cells.

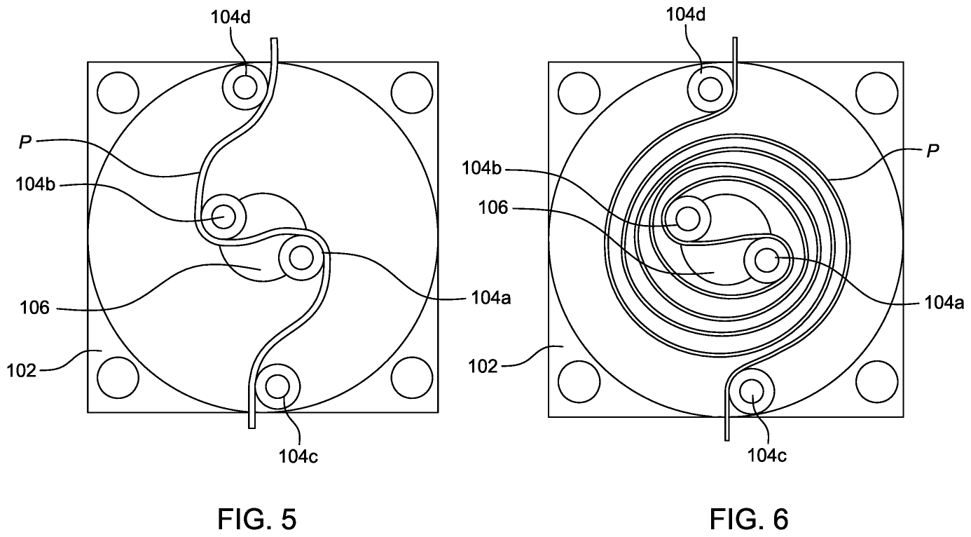

As shown in FIGS. 4-7 and 10-13, the loading device 100 also includes, or be configured to receive in the working space, a portion of tubing 120 having an elongated lumen therethrough. In one embodiment, the portion of tubing 120 may be made of a polymeric or other suitable material and the lumen may have a square cross-sectional shape. Using a tubing with a square shape helps to prevent rolling of the tubing 120 while in use. However, it should be understood that the lumen/tubing 120 may have a circular, oval, or other suitable cross-sectional shape. The portion of tubing 120 may be configured to extend through, around or about the plurality of rollers 104 (for example, rollers 104a, b, c, and d) on the bottom platform 102 to lie in, or define an, adjustable/variable tubing path P (see FIGS. 5 and 6 and 10-13).

Referring now to FIGS. 5 and 6, the tubing path P has a shape that is defined by the portion of tubing 120 positioned in the working space and/or extending between the plurality of rollers (104a-d). More specifically, the tubing 120 can be positioned between a fixed roller (roller 104d) and movable roller (roller 104b), and between both movable rollers (rollers 104b, 104a), and also between the other fixed roller (roller 104c) and the other movable roller (roller 104a). The tubing path P is configured to have a first length and/or a first shape when the movable section 106 of the bottom platform 102 is in the first position (FIGS. 4, 5, 10, and 12) and a second length and/or a second shape, different from the first length and first shape, respectively, when the movable section 106 of the bottom platform 102 is in the second position (FIGS. 6, 11, and 13).

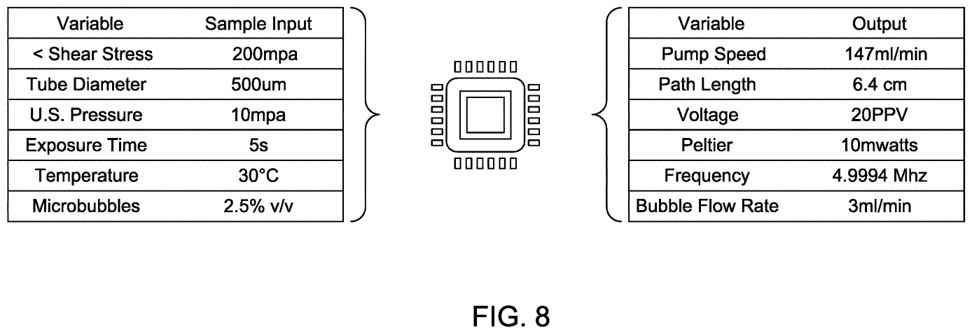

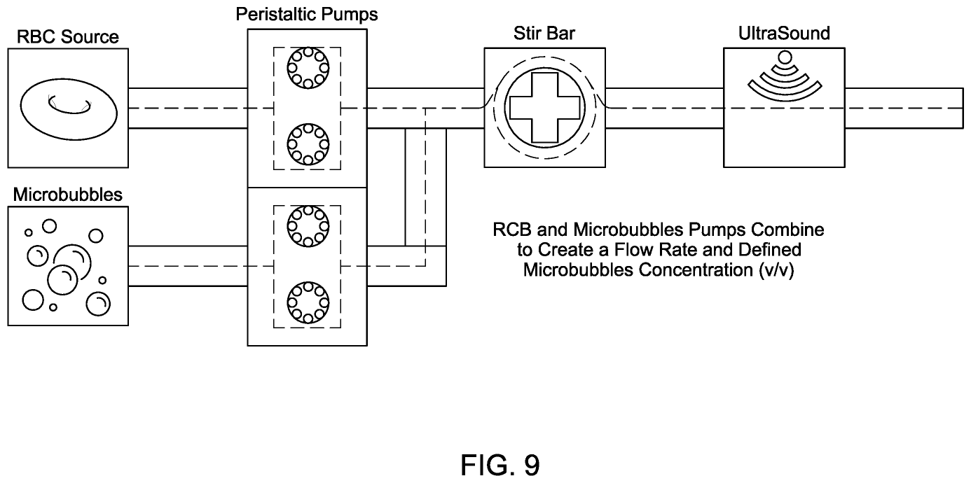

In this embodiment, the movement of the movable section 106 from the first position to the second position is accomplished by rotating the movable section 106, and thereby moving the movable rollers 104a and 104b affixed to the movable portion 106 in a clockwise or counterclockwise motion for a partial, full, or multiple rotation. This movement of the movable section 106 may be accomplished using any suitable means or mechanisms. In one embodiment, the movable portion 106 is rotated manually. In another embodiment, the movement is initiated electronically using a pre-programmed software package configured to rotate the movable portion 106 at a predetermined speed and for a predetermined time, as shown in FIG. 8.

In one embodiment, the upper platform 110 may be moved away from the bottom platform 102, thus increasing the height of the working space, in order to allow the tubing 120 to be moved from the first position to the second position. The height of the working space will typically depend on the size and shape of the tubing. In one embodiment, the height of the working space is increased an amount sufficient to allow the tubing 120 and the rollers to change positions, but not high enough to allow the tubing 120 to slip over the rollers 104. Once the tubing has been successfully moved, the upper platform 110 may be moved back toward the bottom platform 102, thus securing the tubing 120 in place.

As show in FIGS. 5 and 6, the tubing path P may be configured to have a first shape that is an S-shape and a second shape that is a spiral. However, it should be understood that any path shape in which the length of the tubing in the tubing path P changes from the first position to the second position will be acceptable. Those shapes include, but are not limited to spirals, zig-zags, elongated oval shapes, S-shapes, or a combinations thereof. In one embodiment, first length of tubing 120 in the path/working space is less than the second length of tubing in the path.

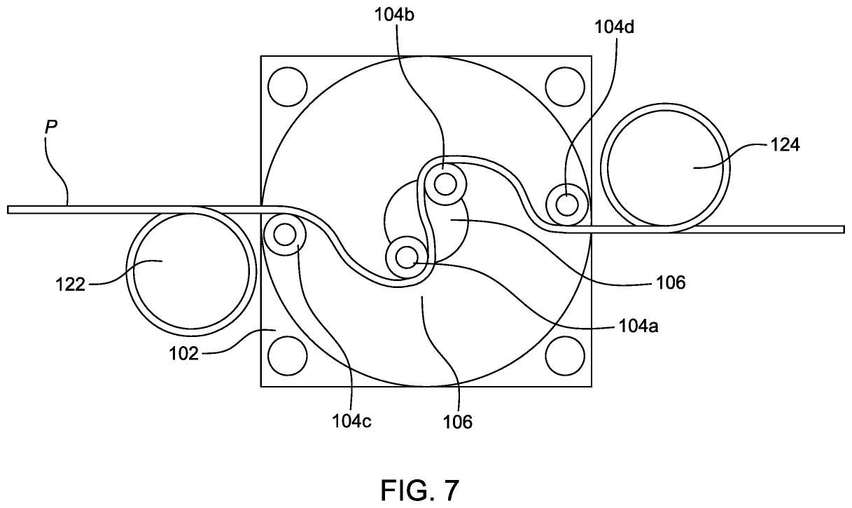

Referring now to FIG. 7, in one embodiment, the loading device 100 further includes a first spooling member 122 disposed a first end of the working space and a second spooling member 124 disposed at a second, opposite end of the working space; wherein the first and second spooling members 122, 124 are positioned and configured to apply and maintain tension of the portion of tubing 120 that is positioned in the working space/extends along the tubing path P. The device 100 may also include a heating or cooling element (not shown) configured to heat and/or cool the portion of tubing 120 and the mixture positioned therein that is positioned in the working space/extending along the tubing path P. The heating/cooling device(s) may be disposed in the stationary portion of the bottom platform 102, or in the spooling members 122, 124, or at other locations, and can take the form of one or more in-line Peltier or thermal sleeve devices, one or more thermal regulators, or a combination of both.

In use, confluent cells 12, such as red blood cells, mixed with osmolytes 14, including CPAs, and also mixed microbubbles 16, are flowed through the tubing 120 which is then exposed ultrasound waves or other energy emitted by the energy emitting device 118 within the resonance frequency of the microbubbles. This emitted energy causes the microbubbles 16 in the mixture to oscillate, eventually resulting in cavitation that produces directional microjets of the surrounding fluid medium that penetrate the membrane of the cells. The microjets cause holes or cavities to be temporarily formed in the cell membranes. Dissolved osmolytes, including CPAs such as trehalose or proline, are thereby forcibly injected and/or naturally flow into the cell 12 through the holes or cavities.

The tubing path P can be changed to accommodate the requirements of different cell types to be treated. Throughput can be optimized by optimizing the path length, tubing size, and flow rate. For example, if more time under the ultrasound device 118 is needed to burst the microbubbles in the mixture, the path length may be increased, while maintaining the flow rate of the solution flowing through the tubing 120. For flow-sensitive cells, a shorter path length may be used to complete cavitation without increasing voltage (i.e. without increasing the time the cells spend subjected to the ultrasound device).

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various elements, these elements should not be limited by these terms. These terms are only used to distinguish one element from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The foregoing description and summary of the invention are to be understood as being in every respect illustrative and exemplary, but not restrictive, and the scope of the invention disclosed herein is not to be determined only from the detailed description of illustrative implementations but according to the full breadth permitted by patent laws. It is to be understood that the implementations shown and described herein are only illustrative of the principles of the present invention and that various modifications may be implemented by those skilled in the art without departing from the scope and spirit of the invention.

1. An adjustable loading device:
a bottom platform comprising a plurality of rollers and at least one movable section; wherein the at least one movable section is configured to move from a first position to a second position;
a portion of tubing having an elongated lumen therethrough; wherein the portion of tubing is configured to extend around or about the plurality of rollers on the bottom platform to create an adjustable tubing path; and
an upper platform disposed above the bottom platform and comprising an energy emitting device that is positioned to emit energy toward at least a portion of the tubing path.

2. The adjustable loading device of claim 1, wherein at least one of the plurality of rollers is fixedly connected to the movable section.

3. The adjustable loading device of claim 1, wherein the loading device is an acoustofluidic, electrofluidic, or magnetofluidic loading device and the energy emitting device is configured to emit ultrasound waves, electric current, or produce a magnetic field.

4. The adjustable loading device of claim 1, wherein the tubing path has a shape that is defined by the portion of tubing extending between the plurality of rollers and is configured to have a first length and a first shape when the movable section of the bottom platform is in the first position and a second length and a second shape when the movable section of the bottom platform is in the second position.

5. The adjustable loading device of claim 4, wherein the tubing path may be configured to have a first shape that is a spiral, zig-zag, elongated oval shape, S-shape, or a combination thereof.

6. The adjustable device of claim 4, wherein the first length is less than the second length.

7. The adjustable loading device of claim 1, wherein the device further comprises at least one threaded pillar configured to connect the bottom platform to the upper platform.

8. The adjustable loading device of claim 7, wherein the upper platform is configured to be movable toward and away from the bottom platform by adjusting the at least one threaded pillar.

9. The adjustable loading device of claim 8, wherein the at least one threaded pillar is connected to a motor that rotates the threaded pillar in a clockwise and counterclockwise direction.

10. The adjustable loading device of claim 1, wherein the device further comprises a first spooling member disposed a first end of the tubing path and a second spooling member disposed at a second end of the tubing path; wherein the first and second spooling members are configured to maintain tension of the portion of tubing that extends along the tubing path.

11. The adjustable loading device of claim 1, wherein the portion of tubing has a square or round cross-section.

12. The adjustable loading device of claim 1, wherein device further comprises a heating element configured to heat the portion of tubing extending along the tubing path.

13. The adjustable loading device of claim 1, wherein device further comprises a cooling element configured to cool the portion of tubing extending along the tubing path.

14. The adjustable loading device of claim 1, wherein movable section of the bottom platform is disposed toward a center portion of the bottom platform and is configured to rotate in a clockwise and counter-clockwise direction.

15. The adjustable loading device of claim 14, wherein the plurality of rollers comprises the at least one roller fixedly connected to the movable section and a second roller fixedly connected to the movable section.

16. The adjustable loading device of claim 15, wherein the movement of the movable section from the first position to the second position is configured to create a spiral shape of the tubing path.

17. An adjustable loading device comprising:
a platform comprising at least two protrusions coupled thereto or positioned adjacent thereto, wherein at least one of the at least two protrusions is movable relative to the platform; and
an energy source positioned adjacent to the platform and configured to direct energy toward the platform; and
wherein the at least two protrusions are configured to receive tubing therebetween such that movement of the at least one movable protrusion adjusts a shape of the tubing.

18. The device of claim 17, wherein the platform includes a plurality of fixed protrusions and a plurality of movable protrusions.

19. The device of claim 17, wherein the movable protrusion is coupled to a movable section.

20. The device of claim 19, wherein the movable section is generally circular, and has an upper surface that is generally flush was an upper surface of the platform.

21. The device of claim 19, wherein the platform includes at least two fixed protrusions positioned at opposite ends thereof and oriented generally perpendicular to an upper surface thereon, and wherein the movable section includes at least two movable protrusions positioned at opposite ends thereof and oriented generally perpendicular to an upper surface thereof.

22. The device of claim 17, further comprising a supplemental platform oriented generally parallel to the platform and defining a working space therebetween, wherein the device is configured such that a distance between the platform and the supplemental platform is adjustable.

23. The device of claim 22, wherein the energy source is at least one of coupled to or embedded in the supplemental platform.

24. The device of claim 17, wherein each protrusion is a roller.

25. The device of claim 17, further comprising the tubing, wherein the tubing is positioned between the at least two protrusions.

26. The device of claim 17, wherein the device is an acoustofluidic, electrofluidic, or magnetofluidic adjustable loading device and the energy emitting device is configured to emit ultrasound waves, electric current, or produce a magnetic field.

27. A method of treating cells comprising:
providing a mixture of cells mixed with microbubbles and at least one cyroprotective agent;
introducing the mixture into a portion of tubing, where at least part of the portion of tubing is located in an adjustable loading device;
providing the adjustable loading device comprising:
a bottom platform comprising a plurality of rollers and at least one movable section; wherein the at least one movable section is configured to move from a first position to a second position; and
an upper platform disposed above the bottom platform and comprising an energy emitting device that is positioned to emit energy toward the bottom platform; and
causing the energy source to emit energy toward the portion of tubing, which is configured to cause the microbubbles in the mixture to oscillate and cause cavitation of cell membranes, such that the at least one cryoprotective agent is introduced into the cells.
